(19) United States
(12) Patent Application Publication
(10) Pub. No.: US 2016/0074551 A1
(43) Pub. Date: Mar. 17, 2016

(54) DISPOSABLE ABSORBENT PAD

(71) Applicant: Carolyn Patrice Smith, Chester Springs, PA (US)

(72) Inventor: Carolyn Patrice Smith, Chester Springs, PA (US)

(21) Appl. No.: 14/690,817

(22) Filed: Apr. 20, 2015

Related U.S. Application Data
(60) Provisional application No. 62/050,934, filed on Sep. 16, 2014.

(51) Int. Cl. A61L 15/52; A61L 15/58; A61F 13/56

(52) U.S. Cl. CPC A61L 15/52; A61F 13/5611; A61F 13/5616; A61L 15/58

(57) ABSTRACT

A menstruation pad member adapted to be used in and out of water is provided. The pad member may provide a plurality of barriers, including a liquid-absorbent core sheet and a liquid-impermeable dressing sheet may be bonded together and sandwiched between a paper backing and an outline structural paper. The paper backing and the structural paper may be removed in use, wherein a user seals the liquid-impermeable dressing sheet about, circumscribing the vagina, with the liquid-absorbent core sheet upward facing, positioned within the vagina.

### CROSS-REFERENCE TO RELATED APPLICATION

This application claims the benefit of priority of U.S. provisional application No. 62/050,934, filed 16 Sep. 2014, the contents of which are herein incorporated by reference.

### BACKGROUND OF THE INVENTION

The present invention relates to disposable absorbent pads and, more particularly, to a disposable menstruation pad adapted to be used in water.

There is currently no adhesive water proof pad available that fits comfortably to a user's vagina so as to prevent menstruation leaks when the user is in the water. For example, when wearing a tampon or menstruation cup when swimming, if a leak occurs, there is nothing between the vagina and bathing suit, allowing the menstruation to soak through the bathing suit. This is because menstruation pads are not designed to be worn in bathing suits, and so they may lose their adhesive properties when wet. In such a state, the current menstruation pad may absorb water rather than the menstruation, leaving a space between the vagina and the menstruation pad to allow for leakage, and possibly falling out of the bathing suit. Moreover, menstruation cups can be uncomfortable, can be intimidating, and can also leak.

Although a few tampons are used for absorption of the menstrual cycle and can be used in the water, they are not ideally used on those lighter days (discomfort upon removal). Moreover, tampons also leak from time to time during normal use, including when full or for those with tilted cervix.

As can be seen, there is a need for a disposable absorbent pad adapted to be used in and out of water. While in the water, the present invention forms a sealed, moisture barrier preventing menstruation and water from soaking through, and while out of the water bolsters the protection of a tampon or menstruation cup, allows for a more comfortable option on the lighter days, and minimizes any fears or anxieties regarding possible leaks.

### SUMMARY OF THE INVENTION

In one aspect of the present invention, a menstruation pad adapted to be used in the water includes a liquid-impermeable dressing sheet forming a dressing periphery; a first adhesive member disposed on the dressing sheet, wherein the first adhesive member has a first perimeter approximately flush with the dressing periphery; at least one side tab disposed along the dressing periphery; and a liquid-absorbent core sheet disposed on a central portion of the first adhesive member, wherein the core sheet has a perimeter smaller than the first perimeter, forming a first adhesive flange.

Another aspect of the present invention is a paper backing having a perimeter approximating the first perimeter; an outline structural paper having a paper periphery forming a paper perimeter approximately matching the dressing perimeter, wherein at least one pull tab is disposed along the paper periphery, and wherein a second adhesive portion is provided near the paper perimeter; and wherein the second adhesive portion removably bonds to the dressing sheet so that the dressing sheet is sandwiched between one side of the first adhesive member and the outline structural paper, and wherein the paper backing sandwiches the core sheet between an opposing side of the first adhesive member.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is an exploded view of an exemplary embodiment of the present invention;

FIG. 3 is a section view of an exemplary embodiment of the present invention, taken along line 3-3 in FIG. 1;

FIG. 4 is a section view of an exemplary embodiment of the present invention, shown in use;

FIG. 5 is an enlarged section view of an exemplary embodiment of the present invention, shown in use;

FIG. 6 is an enlarged section view of an exemplary embodiment of the present invention, shown in use; and

FIG. 7 is an exploded view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a menstruation pad member adapted to be used in and out of water. The pad member may provide a plurality of barriers, including a liquid-absorbent core sheet and a liquid-impermeable dressing sheet may be boned together and sandwiched between a paper backing and an outline structural paper. The paper backing and the structural paper may be removed in use, wherein a user seals the liquid-impermeable dressing sheet about, circumscribing the vagina, with the liquid-absorbent core sheet upward facing, positioned within the vagina.

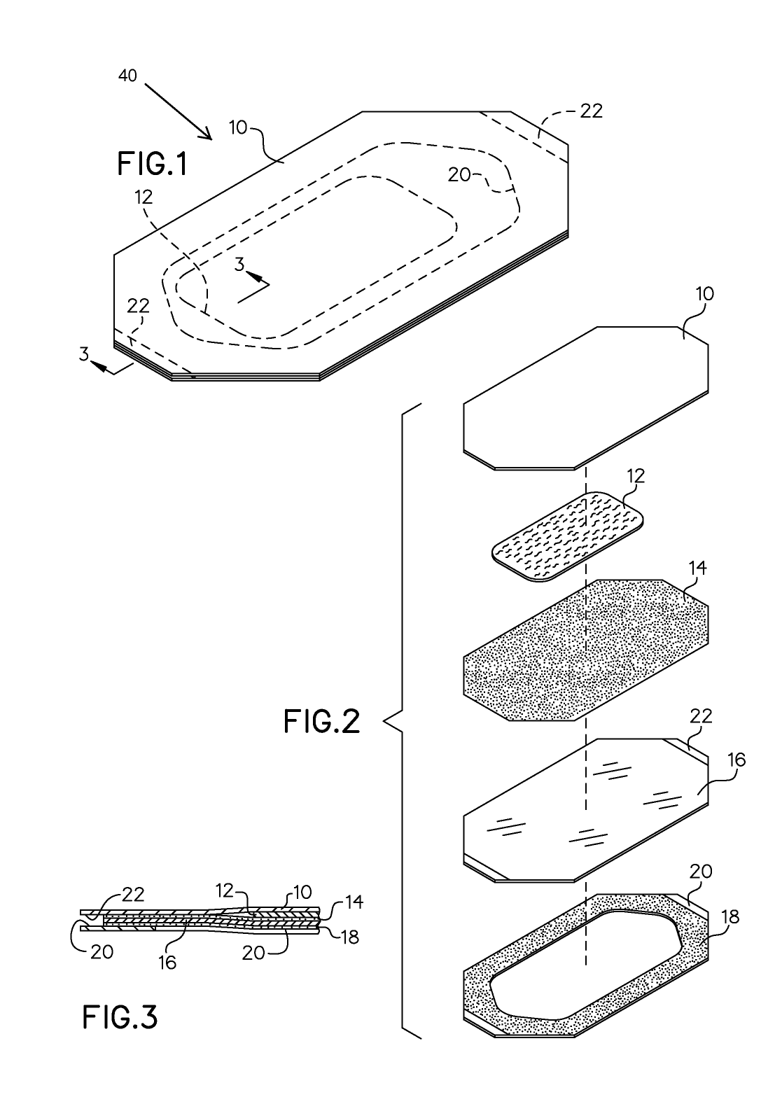

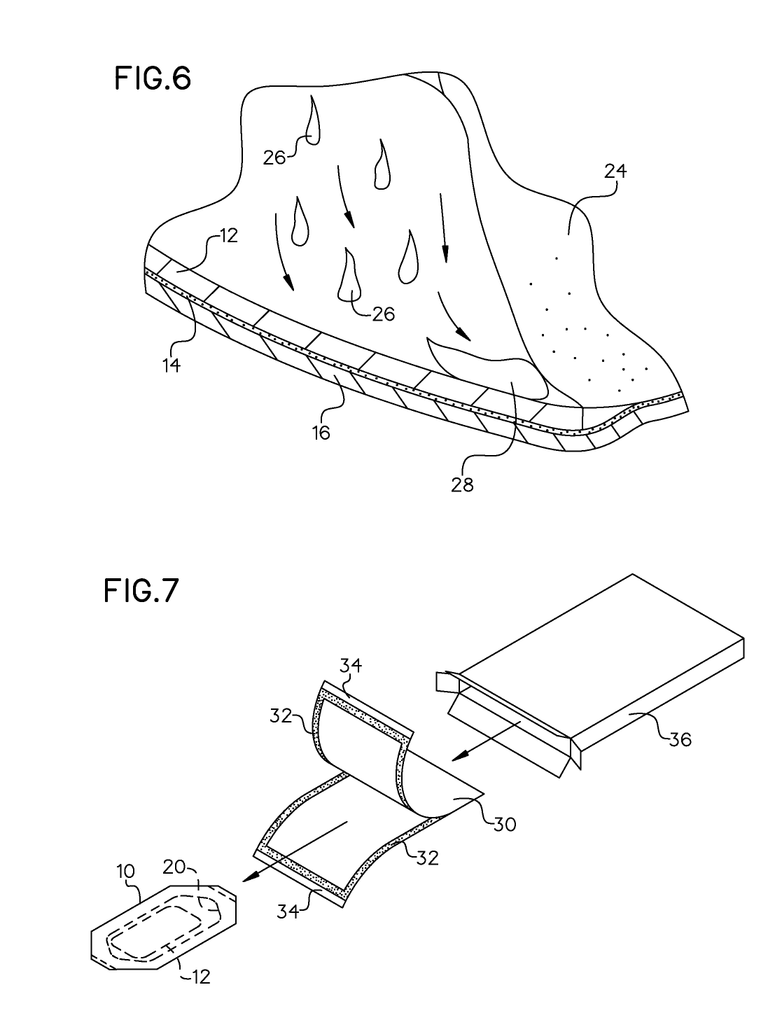

Referring to FIG. 1 through 7, the present invention may include the following components:
10: paper backing
12: liquid-absorbent core sheet
14: first adhesive member
16: dressing sheet
18: adhesive portion
20: outline structural paper
22: side tabs
24: user
26: menstrual blood
28: trapped/contained menstrual blood
30: sanitary wrapper
32: adhesive
34: pull flanges
36: container
40: pad member

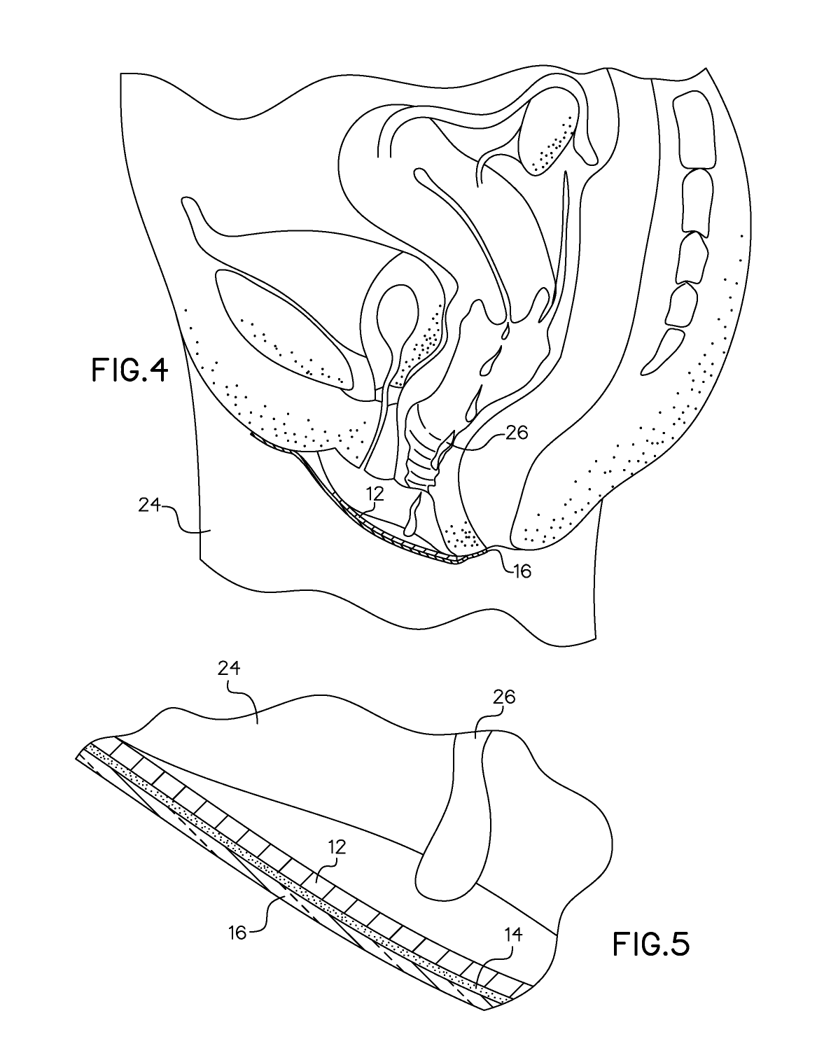

The present invention may include a pad member 40 adapted to be used in and out of water. The pad member 40 may provide a plurality of barriers, including a liquid-absorbent core sheet 12 and a liquid-impermeable dressing sheet 16 sandwiched between a paper backing 10 and an outline structural paper 20, as illustrated in FIGS. 2 and 3.

The liquid-impermeable dressing sheet 16 may include transdermal tegaderm dressing and the like. The dressing sheet 16 may include at least one side tab 22 disposed along a periphery thereof. Each side tab 22 may be adapted for a user 24 to manually pull, peel, rend or the like the dressing sheet 16.

The liquid-absorbent core sheet 12 may be made from fluff pulps, absorption polymer powders, cotton, a combination thereof, or the like.

The outline structural paper 20 may be dimensioned and adapted to maintain the shape of the dressing sheet 16. The dimensions of the outline structural paper 20 may define an opening so that the outline structural paper 20 interfaces with the dressing sheet 16 along the latter's periphery. The outline structural paper 20 may include at least one pull tab disposed along a periphery thereof. Each pull tab may be adapted for the user 24 to manually pull, peel, rend or the like the outline structural paper 20.

A first adhesive member 14 may be disposed on the liquid-impermeable dressing sheet 16 so as to bond the latter to the paper backing 10 and the core sheet 12, as illustrated in FIGS. 2 and 3. The first adhesive member 14 may be made of any suitable transdermal tegaderm adhesive, with two-sided bonding.

The outline structural paper 20 may provide an adhesive portion 18 near a perimeter thereof so as to bond the outline structural paper 20 to the dressing sheet 16 on a side opposing the core sheet 12, as illustrated in FIGS. 2 and 3. The adhesive portion 18 may be made of any suitable adhesive, bonding agent or the like.

The dressing sheet 16 has a dressing periphery forming a two-dimensional perimeter that can be substantially oval, square, triangular or other geometric shape, as well as have angled or rounded corners. The dressing sheet 16 may be designed in various sizes. For example, one size may be defined as approximately 2⅜ inches wide and approximately 5 inches in length. Another size of the dressing sheet 16 may be defined as approximately 5¾ inches in length and approximately 3 inches in width.

The liquid-absorbent core sheet 12 may be centrally positioned and securely attached to the first adhesive member 14. The liquid-absorbent core sheet 12 may be dimensioned, adapted and similarly shaped to cooperate with the dimensions and shape of the first adhesive member 14 so that the periphery of the latter extends outwardly beyond the periphery of the former, forming a first adhesive flange. In certain embodiments, the first adhesive member may have a first perimeter approximately flush with the dressing periphery.

The outline structural paper 20 may be dimensioned, adapted and similarly shaped to cooperate with the peripheral dimensions and shape of the dressing sheet 16 so that the periphery of the outline structural paper 20, and so the adhesive portion 18, may extend outwardly beyond the periphery or the perimeter of the dressing sheet 16, forming a second adhesive flange. In an alternative embodiment, the outline structural paper 20 may be dimensioned, adapted and similarly shaped to be flush with the perimeter and/or periphery of the dressing sheet 16.

The paper backing 10 may be dimensioned, adapted and similarly shaped to cooperate with the peripheral dimensions and shape of the dressing sheet 16, so that the dressing sheet 16 and the paper backing 10 are approximately dimensioned the same, defining an approximately flush alignment of peripheries. In certain embodiments, the outline structural paper 20 may be approximately dimensioned the same and the dressing sheet 16 and the paper backing 10, defining a flush alignment of peripheries.

In certain embodiments, the paper backing 10 may be dimensioned, adapted and similarly shaped to cooperate with the peripheral dimensions and shape of the outline structural paper 20 so that the periphery of the paper backing 10 may extend outwardly beyond the periphery of the outline structural paper 20, forming a first paper flange. The first paper flange and the second adhesive flange may be sized and dimensioned so that at least of a portion of each bond when the liquid-absorbent core sheet 12 and the liquid-impermeable dressing sheet 16 may be sandwiched between the paper backing 10 and the outline structural paper 20.

In certain embodiments, the present invention may include a sanitary wrapper 30 adapted to house the pad member 40. The sanitary wrapper 30 may include of two similarly shaped sanitary sheets, wherein one sheet is slightly longer that the other, forming at least one pull flange 34. Along a portion of the periphery of the two sheets, an adhesive 32 may connect the two sheets, forming a pocket for removably housing the pad member 40 therein until the user 24 pulls at the at least one pull flange 34. In certain embodiments, a container 36 may temporarily house a plurality of the pad member 40, each wrapped by the sanitary wrapper 30, as illustrated in FIG. 7.

A method of using the present invention may include the following. The pad member 40 disclosed above may be provided. After removing any sanitary wrapper 30, the user 24 may pull off the paper backing 10 and discard the backing 10. Then the user 24 may approximately center the liquid-absorbent core sheet 12 up against the vagina, and, in certain embodiments, starting from the bottom (directly above the anus), press the first adhesive flange onto the skin between the anus and labia majora. Keeping the labia majora gently closed for comfort, continue to press the first adhesive flange about, circumscribing the vagina, ensuring that the entire vagina is sealed. Then the user 24, may peel the outlined structural paper 20 off of the dressing sheet 16, by using the at least one pull tab, and gently press the first adhesive flange against the skin near the periphery of the vagina to ensure that the dressing is appropriately secured all around. When in place, the pad member 40 prevents menstrual blood 26, 28 leakage.

The first adhesive member 14/second adhesive portion 18 may be adapted to stick to many textures, including pubic hair and skin, with little to no discomfort upon removal. When ready for removal, the user 24 may grab the at least one side tab 22, and gently pull away from the vagina off of the pubic hair and/or skin. Once the pad member 40 has been used, it will need to be discarded as it is not reusable.

The user 24 could solve the problem of menstruation leakage when in the water by applying the pad member 40 to their vagina before entering the water. It can be used along with the use of a tampon on those heavier days, when tampons have a tendency to leak. The tampon would be placed inside the vagina first and the pad member 40 would be place over the vagina afterwards. It can also be used alone on those lighter days when tampons are not necessary, but there is a threat or chance of possible menstruation. The pad member 40 could be placed onto the vagina prior to entering the water and would remain on the body throughout the length of time that person was in the water, wherein the liquid-impermeable dressing sheet 16 acts as a moisture barrier to keep water out and menstrual blood 26, 28 in. It can remain on the body while out of the water and/or up until the individual has to go to the bathroom or is finished in the water. It will need to be removed prior to urinating and discarded after use. A new pad member 40 would be applied if that individual wanted to reenter the water.

It should be understood, of course, that the foregoing relates to exemplary embodiments of the invention and that modifications may be made without departing from the spirit and scope of the invention as set forth in the following claims.

What is claimed is:

1. A menstruation pad system adapted to be used in the water, comprising:
a liquid-impermeable dressing sheet forming a dressing periphery;
a first adhesive member disposed on the dressing sheet, wherein the first adhesive member has a first perimeter approximately flush with the dressing periphery;
at least one side tab disposed along the dressing periphery;
a liquid-absorbent core sheet disposed on a central portion of the first adhesive member, wherein the core sheet has a perimeter smaller than the first perimeter, forming a first adhesive flange;
a paper backing having a perimeter approximating the first perimeter;
an outline structural paper having a paper periphery forming a paper perimeter approximately matching the dressing perimeter, wherein at least one pull tab is disposed along the paper periphery, and wherein a second adhesive portion is provided near the paper perimeter; and
wherein the second adhesive portion removably bonds to the dressing sheet so that the dressing sheet is sandwiched between one side of the first adhesive member and the outline structural paper, and wherein the paper backing sandwiches the core sheet between an opposing side of the first adhesive member.

2. The menstruation pad system of claim 1, wherein the dressing sheet is made of transdermal tegaderm material.

3. The menstruation pad system of claim 2, wherein the first adhesive member is made of transdermal tegaderm material.

4. A menstruation pad adapted to be used in the water, comprising:
a liquid-impermeable dressing sheet forming a dressing periphery;
a first adhesive member disposed on the dressing sheet, wherein the first adhesive member has a first perimeter approximately flush with the dressing periphery;
at least one side tab disposed along the dressing periphery; and
a liquid-absorbent core sheet disposed on a central portion of the first adhesive member, wherein the core sheet has a perimeter smaller than the first perimeter, forming a first adhesive flange.

5. The menstruation pad system of claim 4, wherein the dressing sheet is made of transdermal tegaderm material.

6. The menstruation pad system of claim 5, wherein the first adhesive member is made of transdermal tegaderm material.

7. The menstruation pad of claim 6, wherein the first perimeter is approximately 2⅜ inches in width and approximately 5 inches in length.

8. The menstruation pad of claim 6, wherein the first perimeter is approximately three inches in width and approximately 5¾ inches n length.

9. A method of preventing menstruation leakage when in the water, comprising the steps of:
providing a menstruation padding comprising: a liquid-impermeable dressing sheet forming a dressing periphery; a first adhesive member disposed on the dressing sheet, wherein the first adhesive member has a first perimeter approximately flush with the dressing periphery; at least one side tab disposed along the dressing periphery; and a liquid-absorbent core sheet disposed on a central portion of the first adhesive member, wherein the core sheet has a perimeter smaller than the first perimeter, forming a first adhesive flange;
approximately centering the core sheet up against a user's vagina;
keeping the vagina's labia majora gently closed;
pressing the first adhesive flange onto a portion of skin immediately surrounding the vagina; and
forming a liquid-tight seal along the portion of skin.

10. The method of claim 7, further including starting the pressing of the first adhesive flange from a region near the user's anus and the labia majora.
